(19) United States
(12) Patent Application Publication
(10) Pub. No.: US 2008/0132775 A1
(43) Pub. Date: Jun. 5, 2008

(54) ULTRASOUND MEASUREMENT TECHNIQUES FOR BONE ANALYSIS

(75) Inventors: Helge E. Engan, Trondheim (NO); Kjell G. Oygarden, Lorenskog (NO)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: ODETECT AS, Lorenskog (NO)

(21) Appl. No.: 11/865,455

(22) Filed: Oct. 1, 2007

Related U.S. Application Data
(60) Provisional application No. 60/827,565, filed on Sep. 29, 2006.

(51) Int. Cl. A61B 5/05; A61B 5/00

(52) U.S. Cl. 600/407; 600/300

(57) ABSTRACT

In some aspects, the present invention provides a method of measuring bone condition using ultrasound waves. Embodiments can involve transmitting an ultrasonic signal through a portion of a bone to be measured to a receiver. The first and second harmonics of the detected signal can then be isolated. A duration difference can then be determined between (i) the detected signal or a first harmonic of the detected signal and (ii) a higher harmonic of the detected signal. Based on that duration difference, material conditions of the bone can be estimated. Methods according to the present invention can be significantly more robust and repeatable than known methods of measuring bone conditions.

### CROSS-REFERENCE TO RELATED APPLICATION

This application claims priority under 35 U.S.C. § 119(e) to U.S. provisional application 60/827,565, filed Sep. 29, 2006, which is hereby incorporated by reference in its entirety.

### FIELD OF THE INVENTION

The invention relates to improved sensing and analysis of ultrasound measurement signals for use as a diagnostic tool in bone analysis.

### BACKGROUND OF THE INVENTION

The field of ultrasound imaging of mammalian physiology is well known and well established. However, the methodology is dominated by certain techniques which have known limitations that are susceptible to improvement or alteration. This technology is known to be used in the imaging of various sites, such as spinal, wrist, knee, cartilaginous areas, and other musculoskeletal locations in mammals, particularly humans. The use of ultrasound for these sites generally is referred to as Quantitative Ultrasound (QUS), and is often in a competitive role with other imaging modalities.

However, there has recently been some interest in using ultrasound in a predictive role for the disease known as osteoporosis. Osteoporosis is a disease of the skeleton in which the amount of calcium present in the bones slowly decreases to the point where the bones become brittle and prone to fracture. In other words, the bone loses density. It is estimated that over 10 million people in the United States suffer from this disease, and 18 million more have low bone mass, placing them at increased risk for this disorder. Osteoporosis is no longer considered a solely age or gender-dependent, and when diagnosed early it can often be treated successfully. In summary, osteoporosis is a major public health problem characterized by significant morbidity, mortality, and economic burden.

The most often used method to estimate bone mass density is based on X-ray absorption methods. A prominent example of this is DXA (Dual Energy X-ray Absorptiometry). A problem with DXA, however, is that its equipment is quite large, meaning that it is essentially stationary. Therefore, other methods involving lighter and/or smaller equipment are often desirable. Such equipment can be easily transported to make it possible to screen a large part of the population in a relatively easy fashion. These other methods should not, however, produce significantly less accurate results than DXA.

One alternative method of estimating bone mass density is based on ultrasound. Ultrasonic signals can be transmitted through a portion of a bone being measured. Some or all of that signal can be detected after transmission through the bone. A linear parameter of the detected signal can be determined. Typical examples of linear parameters of ultrasonic signals include reflection of transmitted sound, scatter of sound, attenuation of sound, speed of sound, broadband ultrasound attenuation, and combinations thereof. Estimating material conditions of a bone based on how the bone impacts the linearity of an ultrasonic signal is well known.

Similarly, some methods of estimating material conditions of a bone based on how the bone impacts the nonlinearity of an ultrasonic signal are known. For example, the amplitude of the first and second harmonics of the detected ultrasonic signal can be determined. These two values can be compared with the transmitted ultrasonic signal. Such a comparison can be used to estimate material conditions of the bone through which the ultrasonic signal was transmitted. This kind of method is covered in commonly assigned U.S. Pat. No. 6,899,680, entitled “Ultrasound Measurement Techniques for Bone Analysis,” which is hereby incorporated by reference herein in relevant part.

### SUMMARY OF THE INVENTION

In some aspects, the present invention provides a method of measuring bone condition using ultrasound waves. Embodiments can involve transmitting an ultrasonic signal through a portion of a bone to be measured to a receiver. The first and second harmonics of the detected signal can then be isolated. A duration difference can then be determined between (i) the detected signal or a first harmonic of the detected signal and (ii) a higher harmonic of the detected signal. Based on that duration difference, material conditions of the bone can be estimated.

Embodiments of the present invention may provide one or more of the following advantages. Methods according to the present invention can be performed by equipment that is significantly smaller and more portable than DXA equipment. Consequently, people who are not able to access facilities that have DXA equipment (e.g., at specialists' offices) can still be tested for osteopenia/osteoporosis (e.g., at primary care providers' offices). Likewise, testing according to some embodiments of the present invention can be significantly less expensive than other methods. Methods according to the present invention can eliminate the risk associated with radiation exposure that is present in DXA processes. Methods according to the present invention can be significantly more robust and repeatable than known methods, including known ultrasound methods, of measuring bone conditions. Tests performed on similar patients under similar conditions often yield similar results under methods according to the present invention. The method may potentially be able to predict and prevent bone fracture (e.g., hip fracture), which could save a substantial amount of money for the health care system and society.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a schematic of ultrasound wave propagations in tissue and bone media.

FIG. 2 is a block diagram of a pulse propagation measuring setup.

FIG. 3 is a block diagram of a backscatter or reflection measuring setup.

FIG. 4 is a block diagram of a reflection at an angle measuring setup.

FIG. 5 is a schematic diagram of a typical experimental set-up.

FIG. 6 is a graph of the amplitude of the second harmonic of the detected signal compared with the amplitude of the transmitted signal or the first harmonic of the detected signal, according to an experiment discussed herein.

FIG. 7 is a graph showing the results shown in FIG. 6 and known T-score values.

FIG. 8 is a graph showing a representative transmitted signal, detected signal, and second harmonic of the detected signal, according to an experiment discussed herein.

FIG. 9 is a graph showing a representative first harmonic and second harmonic of the detected signal, according to an experiment discussed herein.

FIG. 10 is a graph showing results of measurements taken pursuant to an experiment discussed herein.

FIG. 11 is a schematic view showing an ultrasonic transmitter and a receiver positioned proximate to a bone and oriented at an oblique angle to one another.

### DETAILED DESCRIPTION OF ILLUSTRATIVE EMBODIMENTS

The following detailed description is exemplary in nature and is not intended to limit the scope, applicability, or configuration of the invention in any way. Rather, the following description provides practical illustrations for implementing exemplary embodiments of the present invention. Constructions, materials, dimensions, and manufacturing processes suitable for making embodiments of the present invention are known to those of skill in the field of the invention. Those skilled in the art will recognize that many of the examples provided have suitable alternatives that can be utilized.

Osteoporosis is also defined as a skeletal disorder characterized by compromised bone strength predisposing to an increased risk of fracture. Bone strength reflects the integration of two main features: bone density and bone quality. Bone density is expressed as grams of mineral per area or volume and in any given individual is determined by peak bone mass and amount of bone loss. Bone quality refers to architecture, turnover, damage accumulation (e.g., microfractures) and mineralization. Osteoporosis is well established as a significant risk factor for fracture.

Osteoporosis can be further characterized as either primary or secondary. Primary osteoporosis can occur in both genders at all ages but often follows menopause in women and occurs later in life in men. In contrast, secondary osteoporosis is a result of medications, other conditions, or diseases. Osteoporosis is diagnosed when bone density has decreased to the point where fractures will happen with mild stress, its so-called fracture threshold. This is defined by the World Health Organizations as bone mass density (BMD) that is a 2.5 standard deviation (SD) or more below the average BMD for young adults. (One standard deviation below the norm in a measurement of hip bone density is equivalent to adding 14 years to a person's risk for fracture.) Measurements of between 1 and 2.5 SD below normal are defined as osteopenia.

The consequences of osteoporosis include the financial, physical, and psychosocial, which significantly affect the individual as well as the family and community. An osteoporotic fracture is a tragic outcome of a traumatic event in the presence of compromised bone strength, and its incidence is increased by various other risk factors. Traumatic events can range from high-impact falls to normal lifting and bending. The incidence of fracture is high in individuals with osteoporosis and increases with age. Osteoporotic fractures, particularly vertebral fractures, can be associated with chronic disabling pain. Nearly one-third of patients with hip fractures are discharged to nursing homes within the year following a fracture. Notably, one in five patients is no longer living 1 year after sustaining an osteoporotic hip fracture. Hip and vertebral fractures are a problem for women in their late 70s and 80s, wrist fractures are a problem in the late 50s to early 70s, and all other fractures (e.g., pelvic and rib) are a problem throughout postmenopausal years. Indeed, the National Osteoporosis Foundation (United States) estimates that there are more than 1.5 million fractures reported each year.

By way of example, hip fracture alone has a profound impact on quality of life, as evidenced by findings that 80 percent of women older than 75 years preferred death to a bad hip fracture resulting in nursing home placement. However, little data exist on the relationship between fractures and psychological and social well-being. Other quality-of-life issues include adverse effects on physical health (impact of skeletal deformity) and financial resources. An osteoporotic fracture is associated with increased difficulty in activities of daily life, as only one-third of fracture patients regain pre-fracture level of function and one-third require nursing home placement. Fear, anxiety, and depression are frequently reported in women with established osteoporosis and such consequences are likely under-addressed when considering the overall impact of this condition. Direct financial expenditures for treatment of osteoporotic fracture are estimated at $10 to $15 billion annually. A majority of these estimated costs are due to in-patient care but do not include the costs of treatment for individuals without a history of fractures, nor do they include the indirect costs of lost wages or productivity of either the individual or the caregiver.

Currently, the most popular technique for determining bone density is dual-energy x-ray absorptiometry (DXA), which measures bone density throughout the body within two to four minutes. The measurements are made by detecting the extent to which bones absorb photons that are generated by very low-level x-rays. Physicians use a formula based on the results of these procedures to determine if bone density has deteriorated to the fracture threshold.

Unfortunately, DXA is not widely available and may be inappropriate for many patients. Other techniques that measure density may also result in accurate measures of overall bone loss and be less expensive and may not expose the patient to the radiation inherent to DXA and its analogs. These are examples of the opportunities for ultrasound, subject to basic improvements in its accuracy, sensitivity, and overall predictive value.

Use of ultrasound in relation to monitoring of bone growth is also well documented. With respect to bone healing, one study reports that callus (i.e., the hard bonelike substance thrown out between and around the ends of a fractured bone) is easily visualized with ultrasound. Moreover, callus as seen on ultrasound predates its appearance on radiographs. It has also been suggested that fracture union on ultrasound precedes radiographic union. Thus, it is believed that ultrasound may provide important prognostic information concerning fracture healing as well as valuable information of regenerate bone during the process of limb lengthening.

Ultrasound has been used for many years to investigate the mechanical properties of various engineering materials. It offers the theoretic advantage of measuring material properties other than density. As noted above, this technique is termed quantitative ultrasound (QUS). This offers the advantages of small size, relatively quick and simple measurements, and no radiation. QUS measurements are generally considered as much easier to perform at skeletal sites with minimal soft tissue covering. However, to date, most QUS devices measure the peripheral skeleton, including the heel, shin, knee cap, and fingers only, due to certain limitations.

Regardless, several different QUS devices and methods have been shown to be predictive of hip fracture, independent of radiograph-based bone density measurements. QUS has enjoyed widespread use around the world and has recently been approved for clinical use in the United States. Indeed, certain changes in government reimbursement schemes may even accelerate the introduction and use of QUS technologies in order to avail lower cost high quality methodologies to a greater population. Although apparently the QUS technologies are exciting, there are still concerns and room for improvements. For example, researchers are still not certain exactly which mechanical or structural parameters of the bone are being measured with QUS. It has been speculated that QUS may be related to trabecular size, trabecular spacing, and parameters of bone mineralization such as crystal size and orientation.

In yet another analysis, it has been found that broadband ultrasound attenuation (BUA) also predicts the occurrence of fractures in older women and is a useful diagnostic test for osteoporosis. The strength of the association between BUA and fracture is similar to that observed with bone mineral density. Broad-band acoustic attenuation and speed-of-sound have also been shown to display a quantitative relationship to mineralization. Further, in another study, measurements of the attenuation and velocity of ultrasound from 0.3 to 0.8 MHz have been performed on a number of bovine cancellous bone samples. The influence of bone mineral content was isolated by measuring the acoustic properties of the samples at various stages of demineralization resulting from controlled nitric acid attack. The correlation coefficient r, between the attenuation at different frequencies and bone density was found to be in the range 0.68-0.97. Broadband ultrasonic attenuation (BUA) was also calculated and produced r values between 0.84 and 0.99. The velocity measurements indicated a correlation greater than 0.97 in all cases. Thus velocity appears to be the parameter most sensitive to changes in bone mineral density alone. Attenuation and BUA are less well correlated presumably because of a sensitivity to minor structural change. Accordingly, further advances in research are required and encouraged.

Yet another study determined that each standard deviation decrease in calcaneal broadband ultrasound attenuation was associated with a doubling of the risk for hip fractures after adjustment for age and clinic. The relationship was similar for bone mineral density of the calcaneus and femoral neck. Decreased broadband ultrasound attenuation was associated with an increased risk for hip fracture. A low broadband ultrasound attenuation value was particularly strongly correlated with intertrochanteric fractures, i.e., fractures at the proximal femur. The conclusion reached was that decreased broadband ultrasound attenuation predicts the occurrence of fracture in elderly women and that this may also provide a useful diagnostic test for osteoporosis. Thus, the need to accurately account for attenuation and sound velocity profiles of bone in patients at various sites is quite important in this fight against osteoporosis.

In summary, osteoporosis is a major public health problem characterized by significant morbidity, mortality, and economic burden. Osteoporotic fractures in older women are related, for the most part, to the women's BMD. Ultrasound does not measure bone density but rather measures at least two parameters called speed of sound (SOS) and broadband ultrasound attenuation (BUA) that are related to the structural properties of bone. Studies have shown that QUS measures have the ability to distinguish fracture patients from controls and to predict future fracture. Some advantages of ultrasound devices are that they are small, portable, use no ionizing radiation, and may provide an attractive alternative to radiation-based densitometry. Bone mass measurement appears to be one of the best ways to make the diagnosis of osteoporosis. However, considerable improvements are needed in this emerging area of medical technology.

Methods for measuring bone density by ultrasound include measurement of direct transmission and scatter measurements, sending sound through a bone, and measuring acoustic transmission and speed of sound, including reflection. The velocity of sound in bone can be measured using a technique analogous to that used in the field of refraction seismics, which involves investigations of the sea floor for various purposes. As applied to physiological testing, the method includes a first transducer transmitting an ultrasonic wave from a point external of the tissue into an inner bone at a critical angle. This generates pressure, shear and/or surface waves that propagate along the interface between the bone and the soft tissue. The wave radiated from these waves is then received by a second transducer, also positioned external to the tissue. The speed of sound in the bone is calculated from the first time of arrival of the sound pulse at the receiving transducer. This method requires the velocity of sound in bone to be greater than in the surrounding soft tissue, which is true for pressure waves, but may not be fulfilled for shear waves.

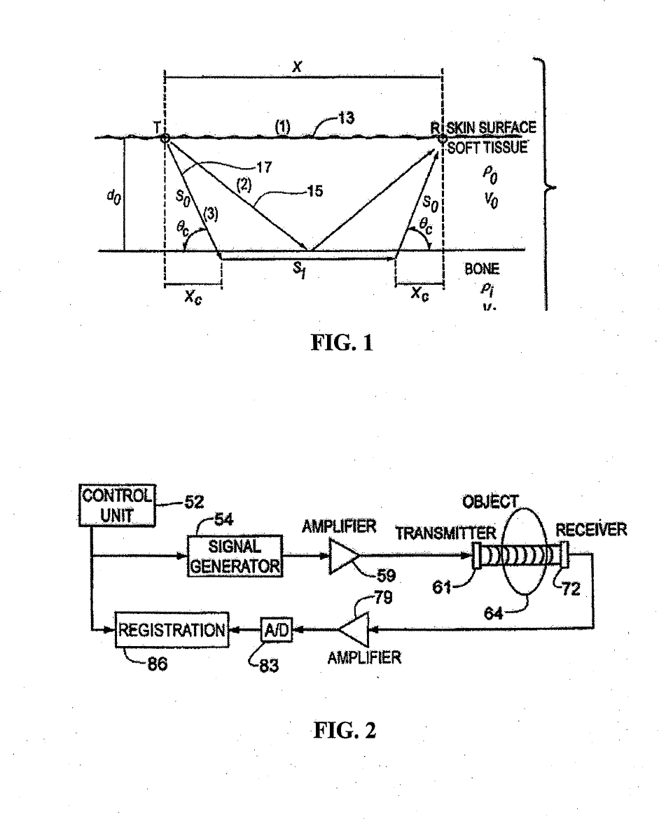

The method is illustrated in FIG. 1, and is summarized as follows. An acoustic wave is emitted from the transmitter T into the body of the patient and received with the receiver R. T and R are placed on the skin of the patient at a distance x. The emitted wave may follow three paths from T to R:
(i) Direct wave. This wave follows a straight line parallel to the skin surface and is denoted by line 13.
(2) Reflected wave. This wave is reflected at the boundary between the soft tissue and the bone, and is denoted by line 15.
(3) Refracted wave. This wave, denoted by line 17, hits the bone at critical angle θ_{c}, propagates along the interface between soft tissue and bone, while radiating acoustic energy back to the tissue at critical angle θ_{c}. Some of the radiated sound is received by the receiver R. The critical angle θ_{c} is given by

$${θ}_{c} = \frac{{υ}_{0}}{{υ}_{1}} \qquad ( 1 )$$

where υ₀ is the speed of sound in the tissue and υ₁ is the speed of sound in the bone.

The time of flight from T to R for these three waves are t₁, t₂ and t₃. The arrival time t₃ of the refracted wave can be found from FIG. 1 to be

$${t}_{3} = x / {v}_{1} + 2 {d}_{0} \frac{\sqrt{{v}_{1}^{2} - {v}_{0}^{2}}}{{v}_{0} {v}_{1}} \qquad ( 2 )$$

where x is the distance between transmitter T and receiver R and d₀ is the distance from the skin surface to the bone, as shown in FIG. 1.

The wave velocity υ₁ of the bone is larger than the wave velocity υ₀ of the soft tissue. If, in addition, the distance x between T and R exceeds a minimum value xₘᵢₙ, the refracted wave 17 may arrive on R before the other waves 13, 15, that is

υ₁>υ₀ and x>xₘᵢₙ=>t₃<t₁, t₂ (3)

Hence, the time t₃ can be found from the first arrival of a signal at R after transmitting from T. When the time of first arrival t₃ is measured, the speed of sound in the bone υ₁ is calculated from (Eq. 2). The speed of sound in the soft tissue υ₀ and the distances x and d₀ must be measured independently. This may be done from ultrasound time-of-flight measurements. This technique allows accurate measurements of sound velocity independent of geometric dimensions. This technique may be combined by one or more of the principles below to increase the accuracy of the estimates of sound velocity.

U.S. Pat. No. 5,197,475 illustrates ultrasound measurement setups using such basic principles of ultrasound pressure wave transmission and/or reflection, particularly as a function of angle. The reference provides very broad but useful description of measurement systems and techniques, and also briefly addresses the concept known as shear wave measurements. Elaborating on that latter concept, and other unknown combinations of techniques, is one of the goals of the present invention.

Shear waves do not propagate far in tissue, but will propagate in solid structures like bone. Moreover, the shear wave velocity is more sensitive to material structure than the pressure wave velocity, in that it differs more strongly between various materials. Hence, the shear wave velocity is a more sensitive parameter than pressure wave velocity for detecting the state of the measured bone.

The pressure cₚ and shear cₛ wave velocities of an elastic solid are given by the expressions

$$Cp = \sqrt{\frac{λ + 2 μ}{ρ}} and Cs = \sqrt{\frac{μ}{ρ}} \qquad ( 4 )$$

where ρ is the density and λ and μ are the Lamé coefficients of the material.

Measurement of the shear wave velocity includes an estimate for the second Lamé coefficient μ, which is the shear modulus of the material. Degradation of a material typically causes a reduction in its density ρ and a reduction in material rigidity, that is, lower values of λ and μ. Measurements of both cₚ and cₛ in (Eq. 4) gives more information about the underlying material properties than measurements of cₚ alone.

If a material undergoes a transition from an elastic solid to a looser porous structure, this causes a larger reduction in the shear modulus μ than in the bulk modulus K=λ+⅔ μ. Hence, independent measurements of cₚ and cₛ, calculating e.g. the ratio cₛ/cₚ, will provide information about the relation between the shear and bulk moduli of the material. This gives information about whether the material is changed from an homogeneous solid into a looser porous structure.

Velocity dispersion is a characteristic property of heterogenous media, especially porous materials. If the bone undergoes a transition from homogeneous to porous, it can also change from non-dispersive to dispersive. Hence, sound velocity dispersion can be used as an indicator of altered tissue material structure. In addition, this technique can reduce the need for an accurate measurement of sound velocity, as it only requires relative measurement of phase velocity as function of frequency, and the technique does not depend on accurate measurements of geometric dimensions. In the case of a heterogenous medium, the phase velocity typically undergoes a change where the wavelength is of the same magnitude as the grain size. This transition may be used as an estimate for “grain size” in a porous material. Velocity dispersion measurements can be combined with measurement of frequency dependent attenuation, to further increase the accuracy of the estimates.

Another aspect of ultrasound imaging relates to nonlinearity. All sound propagation is nonlinear, and will generate harmonics at sufficiently high amplitudes over sufficiently long distances. Small voids or other inhomogeneities can act as nonlinear sources in solid materials, and increase the acoustic nonlinearity parameter. Hence, measurements of the degree of nonlinearity in a material can be used to estimate material conditions. Especially, it may be used to estimate whether the material is changing from a homogeneous to a more heterogenous structure.

The thinning and increased brittleness of the bone structure associated with osteoporosis may increase nonlinear mechanical properties. In addition, a reduction in bone mass may give rise to an increase in soft material such as marrow. This exchange of material may also change the nonlinear mechanical response.

There are several ways to measure the degree of nonlinearity. The most obvious is to transmit a sound pulse through the material and measure the harmonic distortion, i.e. the level at harmonics of the transmitted frequency. Here, the second harmonic is the most natural choice, but also higher harmonics, or combinations of harmonics can be used. Harmonic detection is summarized as

Transmit frequency f_{T}

Receive one or more of the harmonics 2f_{T}, 3f_{T}, 4f_{T}, (5)

Nonlinear frequency mixing may be another method. Two frequencies are transmitted through the sample. This can be done either by two separate transducers, or by exciting one transducer with both frequencies. The transmitted or scattered signals from the material is picked up by another, or the same, transducer. Nonlinear mixing will cause sum- and difference frequencies in the received signals. The level at these sum and/or difference frequencies is an indicator of the condition of the material. Nonlinear frequency mixing is summarized as

Transmit frequencies f₁ and f₂

Receive at sum and/or difference frequencies f₁+f₂, f₁−f₂ (6)

The harmonic and nonlinear frequency mixing techniques may also be combined, i.e. receive at sum and difference frequencies of the harmonics. An example would be

Transmit frequencies f₁ and f₂

Receive at sums and/or differences around harmonics, e.g. 2f₁−f₂, 2f₁+f₂, 3f₁+f₂ (7)

Of particular interest are the nonlinear methods identified herein for detection of micro-cracks or micro-fractures in the human bone. These cracks may act as sources for nonlinear acoustic generation, and therefore the methods identified herein may be considered somewhat analogous to recently developed methods for detecting micro-cracks and other defects in nondestructive testing/evaluation of materials known generally as nonlinear acoustic nondestructive evaluation (NANDE) or nonlinear wave modulation spectroscopy. Measurement of acoustic nonlinearity can therefore be used as an indicator of bone condition.

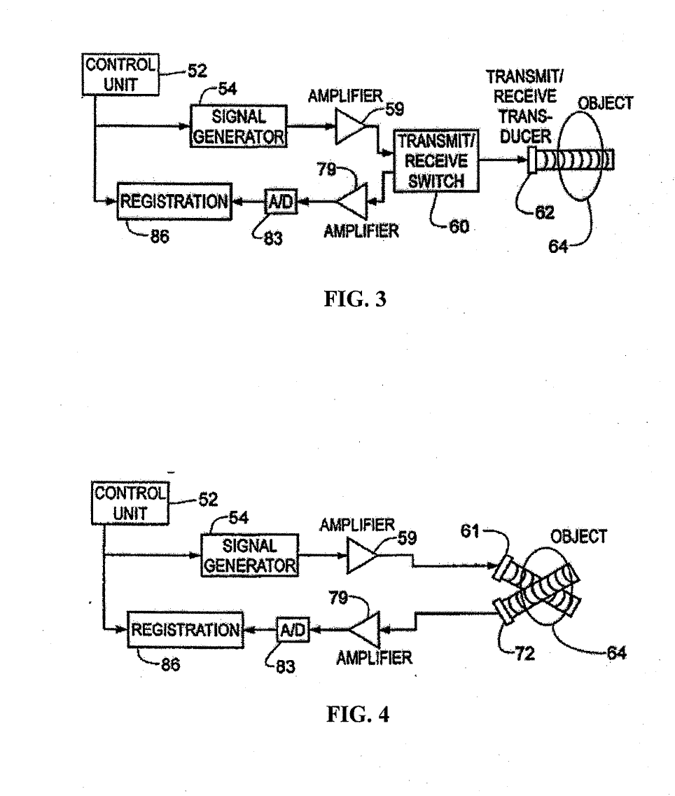

Several of the disclosed measurement methods are considered part of this novel technique. The transmitted signal may either be a continuous wave, CW, or a pulsed wave, PW. The measurements can be accomplished as through-transmission (as shown in FIG. 2), pulse-echo backscatter (as shown in FIG. 3), or scatter at an angle (as shown in FIG. 4). In FIG. 2, there is shown representatively configured components of a control unit 52, signal generator 54, amplifier 59, transmitter 61, the object being measured 64, receiver 72, amplifier 79, analog to digital converter, and registration unit 86. The configuration of FIG. 3 includes most of the similar components but also that of transmit/receive switch 60 and transmit/receive transducer 62. In FIG. 4, the configuration is similar to that depicted in FIG. 2 but with and angled reflection setup. The detection of nonlinearity can be done by any of the following methods:
1. Two frequency mixing by transmitting two frequencies f₁ and f₂. These may then be received at the difference and/or sum frequencies f₁−f₂ and f₁+f₂;
2. Amplitude modulated signal by transmitting a signal p=(1+A sin 2πfₘt) sin 2πf₀t and then receiving at the modulation frequency fₘ and/or its harmonic, e.g., 2fₘ;
3. Transmit one high imaging frequency fᵢ and one low pumping frequency fₚ and then receive at the sum and/or difference frequencies fᵢ−fₚ and fᵢ+fₚ; and
4. Transmit at one frequency f₀ and receive at the harmonics of the transmit frequency, such as 2f₀, 3f₀, 4f₀, . . . or xf₀.

In some embodiments, the present invention provides a method of measuring bone condition using ultrasound waves. The method can include positioning an ultrasonic transmitter and a receiver proximate to a bone. The method can include transmitting an ultrasonic signal from the ultrasonic transmitter through a portion of the bone to the receiver. The method can include detecting at least a portion of the transmitted signal with the receiver after transmission through the bone.

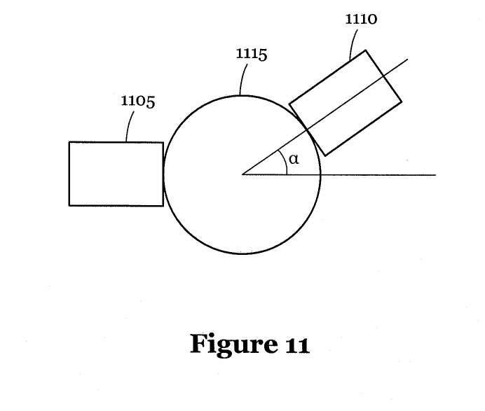

In some embodiments, such as that of FIG. 11, positioning the ultrasonic transmitter 1105 and the receiver 1110 proximate to the bone 1115 can include orienting the ultrasonic transmitter 1105 and the receiver 1110 at an oblique angle (α≠0) to one another. Doing so can cause the detected signal to be composed of an increased percentage of the higher harmonic of the detected signal, as compared with orienting the ultrasonic transmitter in line (α=0) with the receiver. Doing so can also cause the detected signal to be composed of a decreased percentage of the first harmonic of the detected signal, as compared with orienting the ultrasonic transmitter in line with the receiver. This is because the second harmonic is propagated outwardly from the bone 1115 at 360 degrees. When the ultrasonic transmitter 1105 is at an oblique angle to the receiver 1110, the signal to noise ratio of the detected signal is better, but the total detected signal is weaker. Orienting the ultrasonic transmitter 1105 and the receiver 1110 at some oblique angles to one another can result in the detected signal being composed of at least 50% of the higher harmonic of the detected signal. The oblique angle can be at least ±5 degrees; at least ±10 degrees; at least ±20 degrees; at least ±30 degrees; at least ±45 degrees; at least ±60 degrees; at least ±80 degrees; or any other suitable angle.

In some embodiments, the method can include includes determining a duration difference between (i) the detected signal or a first harmonic of the detected signal and (ii) a higher harmonic of the detected signal. Generally, the first harmonic of the detected signal differs only minimally from the entire detected signal. The higher harmonic can be the second harmonic and/or higher harmonics such as the third harmonic, the fourth harmonic, and so on. In many embodiments, determining the duration difference includes comparing (i) an amplitude center of gravity of the detected signal or the detected signal's first harmonic with (ii) an amplitude center of gravity of the detected signal's higher harmonic.

In many embodiments, material conditions of a bone are estimated. In some embodiments, material conditions of the bone are estimated based solely on the duration difference. In some embodiments, material conditions of the bone are estimated based on the duration difference and on other factors. In some such embodiments, material conditions of the bone are estimated based on the duration difference and a comparison of the amplitude of the second harmonic of the detected signal with the amplitude of the transmitted signal or the first harmonic of the detected signal. In some embodiments, material conditions of the bone are estimated based on the duration difference and a linear parameter of the detected signal. Examples of linear parameters include (i) reflection of sound, (ii) scatter of sound, (iii) attenuation of sound, (iv) speed of sound, (v) broadband ultrasound attenuation, and (vi) combinations thereof. In some embodiments, material conditions of the bone are estimated based on the linear parameter, the duration difference, and the comparison of the amplitude of the second harmonic of the detected signal with the amplitude of the transmitted signal or the first harmonic of the detected signal.

The method can be performed in a variety of ways. In some embodiments, wherein the method is performed (a) when the bone is bearing weight and (b) when the bone is bearing negligible weight. In such embodiments, the material conditions of the bone can be estimated when the bone is bearing weight and when the bone is bearing negligible weight, and the results can be compared.

### EXPERIMENT

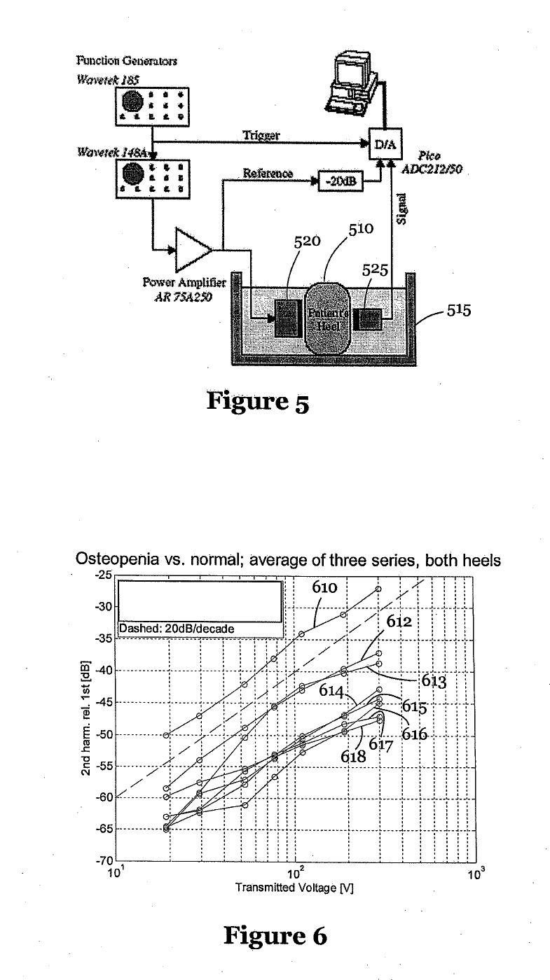

FIG. 5 shows a typical experimental set-up. Seven persons with known T-score values (obtained by DXA) were selected. Based on their T-Score values, two persons were osteopenic and five persons were healthy. Each person's heels 510 were submerged in a water bath 515 (one heel at a time). An ultrasonic signal was transmitted from the transmitter 520 to the receiver 525 through a portion of the heel bone 510. The transmitter 520 was optimized for the fundamental frequency of 236 kHz, eliminating any harmonics from the transmitted signal. The fundamental frequency of 236 kHz is in reasonable agreement with the relevant field. This signal was transmitted through the person's two heels 510 seven times, each with a different voltage (ranging from twenty volts to three-hundred volts). Once these fourteen measurements were completed, the process was repeated twice (i.e., two more signal transmissions at each voltage level).

The receiver detected at least a portion of each transmitted signal. The receiver was a broadband type, covering both the first and second harmonic frequencies. The detected signal was analyzed for frequency contents. The first and second harmonics the detected signal was determined.

Two comparisons were made with the first and second harmonics. First, the amplitude of the second harmonic of the detected signal was compared with the amplitude of the transmitted signal or the first harmonic of the detected signal. As is mentioned above, the transmitted signal was essentially the same as the first harmonic of the detected signal.

FIG. 6 shows how these quantities compared. Line 610 represents the reference values of the water. Lines 612-618 represent the amplitude of the second harmonic of the detected signal relative to the amplitude of the transmitted signal or the first harmonic of the detected signal (measured in dB) for the seven persons (with the three signal transmissions at each voltage level being averaged in the logarithmic regime). Lines 612-613 represent the two osteopenic persons, while lines 614-618 represent the five healthy patients.

Ideally (in water and for small amplitudes) the amplitude of the second harmonic of the detected signal should be proportional to the transmitted signal amplitude. In the higher amplitude regions, there is a significant correlation between the osteoporotic state and the comparison of these two values. The difference between the amplitude of the second harmonic of the detected signal and the transmitted signal amplitude is significantly greater for osteopenic persons than for healthy persons.

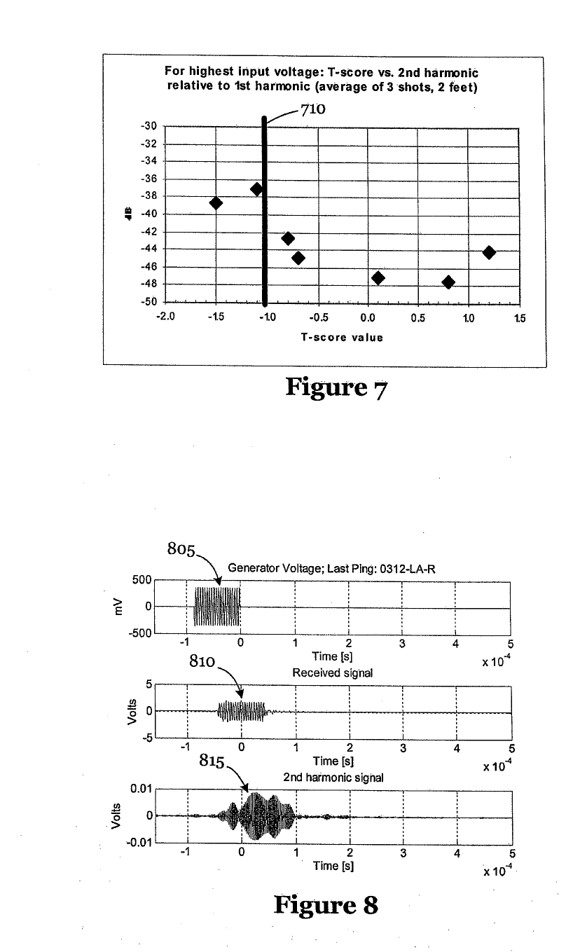

FIG. 7 shows a more detailed comparison of the results shown in FIG. 6 and known T-score values. Referring again to FIG. 7, the difference between the amplitude of the second harmonic of the detected signal and the transmitted signal amplitude at the highest tested voltage is compared with the known T-score values. A thick vertical line 710 is shown at T-score value −1, which is the commonly understood limit between persons with healthy bone structure and persons suffering from osteopenia/osteoporosis. As can be seen, the two persons for whom the difference between the amplitude of the second harmonic of the detected signal and the transmitted signal amplitude is greatest have T-score values less than −1.

Comparing the transmitted signal amplitude with the amplitude of the second harmonic of the detected signal is an evaluation of the amount of energy at the second harmonic being generated and transmitted through the bone. Because the transmitted signal contained no second harmonic component, all of the detected signal's second harmonic component can be attributed to being generated within the bone. This is basically an effect where the second harmonic amplitude is proportional to the square of the transmitted signal's amplitude. This correlation has been verified experimentally.

The second comparison made with the first and second harmonics was determining a duration difference between the first and second harmonics of the detected signal. The envelopes of the first and second harmonics of the detected signal were of different shape, and they differ from person to person. In many cases, the duration of the second harmonic differed from that of the first harmonic (or the entire detected signal). This comparison was designed to determine whether the envelopes correlated to the osteoporotic state (i.e., T-score value).

FIG. 8 shows a representative transmitted signal 805, detected signal 810, and second harmonic of the detected signal 815. The first harmonic of the detected signal, which was essentially identical to the detected signal 810, was generally a slightly modified and delayed version of the transmitted signal 805. The second harmonic of the detected signal 815 was obtained by first Fourier transforming the received signal and selecting the appropriate frequency range, followed by an inverse Fourier transform.

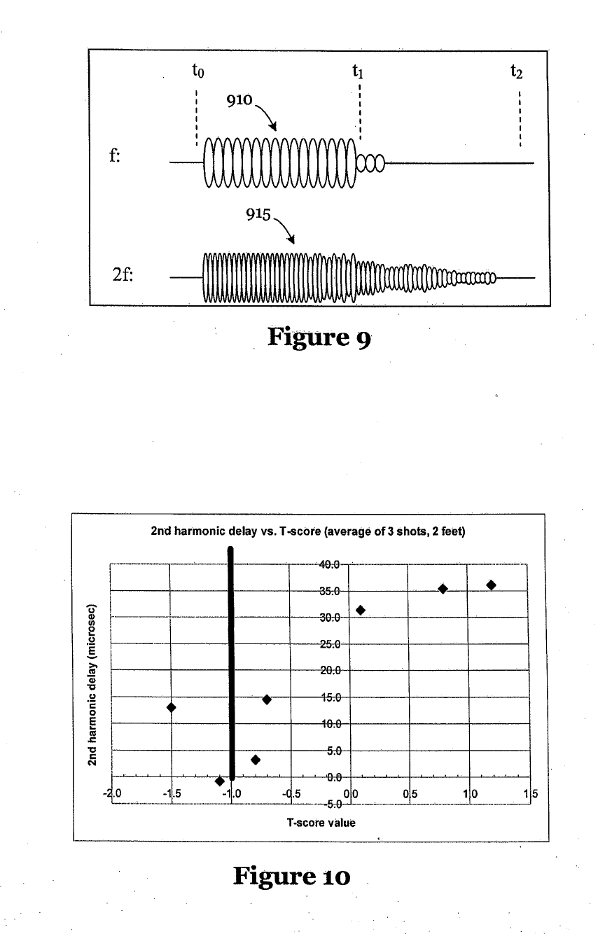

FIG. 9 shows a representative first harmonic 910 and second harmonic 915 of the detected signal. The time at which the first harmonic 910 is first detected is represented as to, the time at which the first harmonic 910 drops off substantially is represented as t₁, and the time at which both harmonics 910, 915 have ceased is represented as t₂. For both the first harmonic 910 and second harmonic 915 of the detected signal, at least some signal arrives after t₁. Two prominent differences were observed between the first harmonic 910 and second harmonic 915 of the detected signal. First, the envelope between t₁ and t₀ of the first harmonic 910 is shaped differently than that of the second harmonic 915. Second, the “tail” of the signal that arrives after t₁ is substantially longer for the second harmonic 915 than for the first harmonic 910.

To determine a duration difference between the first harmonic 910 and the second harmonic 915, a first instant was determined that represented the first harmonic 910 and a second instant was determined that represented the second harmonic 915. The duration difference between the first harmonic 910 and the second harmonic 915 then became the difference (in time) between the first instant and the second instant.

There are a variety of ways to determine the first and second instants. The chosen way was to determine the center of gravity of the two amplitude distributions. The first instant corresponded to the amplitude center of gravity of the detected signal's first harmonic 910. The second instant corresponded to the amplitude center of gravity of the detected signal's second harmonic 915. The amplitude centers of gravity can be determined according to the following formula:

$$Δ τ = {τ}_{2} - {τ}_{1} , with$$

$${τ}_{1} = \frac{{\int }_{{t}_{1}}^{{t}_{2}} \vert {A}_{1} \vert t d t}{{\int }_{{t}_{1}}^{{t}_{2}} \vert {A}_{1} \vert d t} , and {τ}_{2} = \frac{{\int }_{{t}_{1}}^{{t}_{2}} \vert {A}_{2} \vert t d t}{{\int }_{{t}_{1}}^{{t}_{2}} \vert {A}_{2} \vert d t} ,$$

where A₁ and τ₁ are the amplitudes and centers of gravity of the first harmonic 910, and A₂ and τ₂ are the amplitudes and centers of gravity of the second harmonic 915. The range of integration is determined by two reasonably chosen limits t₁ and t₂. Accordingly, the first instant was determined to be the time at τ₁, and the second instant was determined to be the time at τ₂. The duration difference between the first harmonic 910 and the second harmonic 915 was then the difference (in time) between the first instant and the second instant.

There are a variety of other ways to determine a duration difference between the first harmonic 910 and the second harmonic 915. For example, first and second instants can be determined based on energy centers of gravity, rather than amplitude centers of gravity, of the harmonics. Energy centers of gravity can be determined by the following formula:

$$Δ τ = {τ}_{2} - {τ}_{1} , with$$

$${τ}_{1} = \frac{{\int }_{{t}_{0}}^{{t}_{2}} {\vert {A}_{1} \vert}^{2} t d t}{{\int }_{{t}_{0}}^{{t}_{2}} {\vert {A}_{1} \vert}^{2} d t} , and {τ}_{2} = \frac{{\int }_{{t}_{0}}^{{t}_{2}} {\vert {A}_{2} \vert}^{2} t d t}{{\int }_{{t}_{0}}^{{t}_{2}} {\vert {A}_{2} \vert}^{2} d t} ,$$

where A₁ and τ₁ are the amplitudes and centers of gravity of the first harmonic 910, and A₂ and τ₂ are the amplitudes and centers of gravity of the second harmonic 915.

Another example involves comparing the size of the “tail” (between t₁ and t₂) with the size of the main part (between t₀ and t₁) for both the first harmonic 910 and the second harmonic 915. The amplitude centers of gravity of the tail and main part can be determined by the following formula:

$${R}_{1} = \frac{{\int }_{{t}_{1}}^{{t}_{2}} \vert {A}_{1} \vert d t}{{\int }_{{t}_{0}}^{{t}_{1}} \vert {A}_{1} \vert d t} and {R}_{2} = \frac{{\int }_{{t}_{1}}^{{t}_{2}} \vert {A}_{2} \vert d t}{{\int }_{{t}_{0}}^{{t}_{1}} \vert {A}_{2} \vert d t} ,$$

where R₁ is the ratio of the tail to the main part for the first harmonic 910, and R₂ is the ratio of the tail to the main part for the second harmonic 915. The energy centers of gravity for the tail and main part can be determined by the following formula:

$${R}_{1} = \frac{{\int }_{{t}_{1}}^{{t}_{2}} {\vert {A}_{1} \vert}^{2} d t}{{\int }_{{t}_{0}}^{{t}_{1}} {\vert {A}_{1} \vert}^{2} d t} and {R}_{2} = \frac{{\int }_{{t}_{1}}^{{t}_{2}} {\vert {A}_{2} \vert}^{2} d t}{{\int }_{{t}_{0}}^{{t}_{1}} {\vert {A}_{2} \vert}^{2} d t} ,$$

where, again, R₁ is the ratio of the tail to the main part for the first harmonic 910, and R₂ is the ratio of the tail to the main part for the second harmonic 915.

Another example involves the group delay difference based on FFT/frequency information. Based on Fourier transforms of the signal, variations of the phase delay or the group delay as functions of frequency should be distinctly different for pulses propagating through bone structures having varying degree of osteoporosis. This approach is expected to bring interesting results which may enhance the distinction between the various states of osteoporosis.

Another example involves correlating amplitude envelopes of the entire first harmonic and the entire second harmonic. This correlation is given by the following normalized overlap integral (phase relations may be important so “true” envelopes may be needed):

$$C = \frac{{\int }_{{t}_{0}}^{{t}_{2}} \vert {A}_{1} \vert \vert {A}_{2} \vert d t}{\sqrt{{\int }_{{t}_{0}}^{{t}_{2}} {\vert {A}_{1} \vert}^{2} d t {\int }_{{t}_{0}}^{{t}_{2}} {\vert {A}_{2} \vert}^{2} d t}} .$$

This is a criterion which may be used when the other criteria discussed above for some reason are not feasible.

FIG. 10 shows the result of these measurements, where the calculated delay is plotted versus the known T-score values. The delay was averaged over three signals and over both feet. As is shown, there is a variation in the measured parameter that is particularly noticeable in approximately the same range of T-score values as before—around −1 to −0.5.

Duration differences between the first and second harmonics of the detected signal were interpreted as an extra transmission time spent by the second harmonic compared to the first harmonic. Having calculated the duration differences based on the first and second instants, which represented the first and second harmonics, respectively, a quantitative correlation with the known T-score values was performed. This quantity is believed to be associated primarily with scattering processes in the bone structures, although modified by generation and attenuation processes.

The results of the two comparisons of the first and second harmonics show a pronounced correlation between the osteoporotic state and the measured second harmonic amplitude relative to that of the transmitted signal. Also, there is a correlation between the osteoporotic state and the duration difference introduced. For healthy patients, the difference between the duration of the second harmonic and the duration of the first harmonic was significantly greater than for osteoporotic patients. The observed correlations indicate that there is a possibility to use one or two of the methods to discriminate between people of different osteoporosis categories.

It is not clear in detail which mechanisms are responsible for the observed variations. A few possible effects are mentioned here. These include the variation of scattering from the inner trabecular bone as well as the outer cortical bone. Further, such effects can be caused by variation of reflection from the outer, more solid bone part. Also, one source may be variation in the ability to generate second harmonic when the amount of fluid marrow or thin trabecular bone walls varies with the degree of osteoporosis.

The methods mentioned above may be combined in various measuring or display techniques to increase the quality of the outcomes. Further, these techniques may be combined with other measurement techniques, such as measurements of reflection, scatter, attenuation and speed of sound. They may also be combined with estimates for elastic properties, and with measurements of shape and geometrical dimensions.

The invention thus recognizes alternate methods and techniques to improve the quality and availability of ultrasound quantitative measurement modalities for various bone conditions. It is recognized that the various techniques may be combined with or substituted for known techniques and systems to achieve an overall improvement in this measurement capability.

What is claimed is:

1. A method of measuring bone condition using ultrasound waves, comprising the steps of:
(a) positioning an ultrasonic transmitter proximate to a bone;
(b) positioning a receiver proximate to the bone;
(c) transmitting an ultrasonic signal from the ultrasonic transmitter through a portion of the bone to the receiver;
(d) detecting at least a portion of the transmitted signal with the receiver after transmission through the bone;
(e) determining a duration difference between (i) the detected signal or a first harmonic of the detected signal and (ii) a higher harmonic of the detected signal; and
(f) estimating material conditions of the bone based on the duration difference.

2. The method of claim 1, wherein the higher harmonic comprises a second harmonic.

3. The method of claim 1, wherein the higher harmonic includes a harmonic higher than a second harmonic.

4. The method of claim 1, wherein determining the duration difference comprises comparing (i) an amplitude center of gravity of the detected signal or the detected signal's first harmonic with (ii) an amplitude center of gravity of the detected signal's higher harmonic.

5. The method of claim 1, further comprising comparing an amplitude of the transmitted signal or of the first harmonic of the detected signal with an amplitude of the higher harmonic of the detected signal,
wherein estimating material conditions of the bone is based on both the duration difference and the comparison of the amplitude of the transmitted signal or of the first harmonic of the detected signal with the amplitude of the higher harmonic of the detected signal.

6. The method of claim 1, further comprising determining a linear parameter of the detected signal, the linear parameter selected from a group consisting of:
(i) reflection of sound,
(ii) scatter of sound,
(iii) attenuation of sound,
(iv) speed of sound,
(v) broadband ultrasound attenuation, and
(vi) combinations thereof,
wherein estimating material conditions of the bone is based on both the duration difference and the linear parameter.

7. The method of claim 1, wherein the method is performed (a) when the bone is bearing weight and (b) when the bone is bearing negligible weight, and
further comprising comparing estimated bone material conditions when the bone is bearing weight with estimated bone material conditions when the bone is bearing negligible weight.

8. The method of claim 1, wherein positioning the ultrasonic transmitter and the receiver proximate to the bone comprises orienting the ultrasonic transmitter and the receiver at an oblique angle to one another, thereby causing the detected signal to be composed of an increased percentage of the higher harmonic of the detected signal and a decreased percentage of the first harmonic of the detected signal, as compared with orienting the ultrasonic transmitter in line with the receiver.

9. The method of claim 8, wherein the detected signal is composed of at least 50% of the higher harmonic of the detected signal.

10. A method of measuring bone condition using ultrasound waves, comprising the steps of:
(a) positioning an ultrasonic transmitter proximate to a bone;
(b) positioning a receiver proximate to the bone;
(c) transmitting an ultrasonic signal from the ultrasonic transmitter through a portion of the bone to the receiver;
(d) detecting at least a portion of the transmitted signal with the receiver after transmission through the bone;
(c) determining a duration difference between (i) the detected signal or a first harmonic of the detected signal and (ii) a second harmonic of the detected signal; and
(f) estimating material conditions of the bone based on the duration difference.

11. The method of claim 10, wherein determining the duration difference comprises comparing (i) an amplitude center of gravity of the detected signal or the first harmonic with (ii) an amplitude center of gravity of the second harmonic.

12. The method of claim 10, further comprising comparing an amplitude of the transmitted signal or of the first harmonic of the detected signal with an amplitude of the second harmonic of the detected signal,
wherein estimating material conditions of the bone is based on both the duration difference and the comparison of the amplitude of the transmitted signal or of the first harmonic of the detected signal with the amplitude of the second harmonic of the detected signal.

13. The method of claim 10, further comprising determining a linear parameter of the detected signal, the linear parameter selected from a group consisting of:
(i) reflection of sound,
(ii) scatter of sound,
(iii) attenuation of sound,
(iv) speed of sound,
(v) broadband ultrasound attenuation, and
(vi) combinations thereof,
wherein estimating material conditions of the bone is based on both the duration difference and the linear parameter.

14. The method of claim 10, wherein the method is performed (a) when the bone is bearing weight and (b) when the bone is bearing negligible weight, and
further comprising comparing estimated bone material conditions when the bone is bearing weight with estimated bone material conditions when the bone is bearing negligible weight.

15. The method of claim 10, wherein positioning the ultrasonic transmitter and the receiver proximate to the bone comprises orienting the ultrasonic transmitter and the receiver at an oblique angle to one another, thereby causing the detected signal to be composed of an increased percentage of the second harmonic of the detected signal and a decreased percentage of the first harmonic of the detected signal, as compared with orienting the ultrasonic transmitter in line with the receiver.

16. The method of claim 15, wherein the detected signal is composed of at least 50% of the second harmonic of the detected signal.

17. A method of measuring bone condition using ultrasound waves, comprising the steps of:
(a) positioning an ultrasonic transmitter proximate to a bone;
(b) positioning a receiver proximate to the bone;
(c) transmitting an ultrasonic signal from the ultrasonic transmitter through a portion of the bone to the receiver;
(d) detecting at least a portion of the transmitted signal with the receiver after transmission through the bone;
(e) determining a duration difference between (i) the detected signal or a first harmonic of the detected signal and (ii) a higher harmonic of the detected signal;
(f) comparing an amplitude of the transmitted signal or of the first harmonic of the detected signal with an amplitude of the second harmonic of the detected signal; and
(g) estimating material conditions of the bone based on the duration difference and the comparison of the amplitude of the transmitted signal or of the first harmonic of the detected signal with the amplitude of the second harmonic of the detected signal.

18. The method of claim 17, wherein the higher harmonic of the detected signal includes a harmonic higher than a second harmonic.

19. The method of claim 17, wherein determining the duration difference comprises comparing (i) an amplitude center of gravity of the detected signal or the detected signal's first harmonic with (ii) an amplitude center of gravity of the detected signal's higher harmonic.

20. The method of claim 17, further comprising determining a linear parameter of the detected signal, the linear parameter selected from a group consisting of:
(i) reflection of sound,
(ii) scatter of sound,
(iii) attenuation of sound,
(iv) speed of sound,
(v) broadband ultrasound attenuation, and
(vi) combinations thereof,
wherein estimating material conditions of the bone is based on the duration difference, the comparison of the transmitted signal with the detected signal amplitude ratio, and the linear parameter.

21. The method of claim 17, wherein the method is performed (a) when the bone is bearing weight and (b) when the bone is bearing negligible weight, and
further comprising comparing estimated bone material conditions when the bone is bearing weight with estimated bone material conditions when the bone is bearing negligible weight.

22. The method of claim 17, wherein positioning the ultrasonic transmitter and the receiver proximate to the bone comprises orienting the ultrasonic transmitter and the receiver at an oblique angle to one another, thereby causing the detected signal to be composed of an increased percentage of the second harmonic of the detected signal and a decreased percentage of the first harmonic of the detected signal, as compared with orienting the ultrasonic transmitter in line with the receiver.

23. The method of claim 22, wherein the detected signal is composed of at least 50% of the second harmonic of the detected signal.
